(19) United States
(12) Patent Application Publication
(10) Pub. No.: US 2014/0088063 A1
(43) Pub. Date: Mar. 27, 2014

(54) AGENTS FOR TREATING NEURODEGENERATIVE DISORDERS

(71) Applicant: Pharmdiscovery LLP, London (GB)

(72) Inventors: Nina Nikolayevna Makhova, Moscow (RU); Vera Yur'evna Petukhova, Moscow (RU); Alexander Vladimirovich Shevtsov, Serpuhov (RU); Vladimir Vladimirovich Novakovskiy, St. Petersburg (RU)

(73) Assignee: Pharmdiscovery LLP, London (GB)

(21) Appl. No.: 14/089,545

(22) Filed: Nov. 25, 2013

Related U.S. Application Data
(63) Continuation of application No. 13/877,107.
(60) Provisional application No. 61/387,844, filed on Sep. 29, 2010.

(51) Int. Cl. C07D 229/02

(52) U.S. Cl. CPC C07D 229/02
USPC 514/183; 548/960

(57) ABSTRACT

The present invention relates to compounds of formula (I) or (I′), use of these compounds to treat mental disorders, especially depressions of different etiology, and methods for their preparation. The compounds that are provided for the treatment of mental disorders can be presented by a general formula (I) as meso-form—S,2S,1′R,2′R-1-[ω-(3,3-dialkyldiaziridin-1-yl)alkyl]-3,3-dialkyldiaziridines:

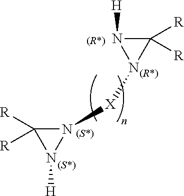

wherein R, X and n are as defined in specification or pharmaceutically acceptable salts thereof, solvates thereof such as hydrates, and pharmaceutical compositions containing such compounds, with the proviso that the compound of Formula (I) is not meso-1S,2S,1′R,2′R-1-[2-(3,3-dimethyldiaziridin-1-yl)ethyl]-3,3-dimethyldiaziridine (R═Me, X═CH2, n=2) (Formula I′).

### BACKGROUND OF THE INVENTION

The present invention relates to compounds of formula (I) and (I′) as meso-form, their pharmaceutically acceptable salts, pharmaceutical compositions containing them, their use for the treatment of mental disorders, especially different depressions, and the methods for their preparation.

The depressions are now one of the widespread forms of psychiatric disorders. The depressive states also accompany many of the mental and somatic distresses. The large number of antidepressant medicines that can be used to treat these conditions ranges over a wide number of antidepressants of different classes. However, the antidepressants can be subdivided into two main groups: 1) inhibitors of monoaminooxydaze (MAO) (e.g. nialamidum—derivative of isonicotin acid, pyrazidole derivative of indole) and 2) inhibitors of neuronal receptor of monoamine neurotransmitters (noradrenaline, dopamine, serotonin) (e.g. imipraminum—derivative of iminodibenzyl class of tricyclic antidepressant). There are also “nontypic” antidepressants having mixed mechanism of action.

The general property of all antidepressants is their positive influence on affective sphere of patients accompanied by an improvement in mood and common mental state. However, a diversity of the pathogenetic forms of diseases demands individual selection of the drugs. Meanwhile the majority of antidepressants have been shown to have more or less severe side effects. Thus tricyclic antidepressants including imipramine, desipramine, nortrypline, amitrypline, doxepin and protryplin produce a variety of anticholinergic side effects, drowsiness, orthostatic hypotension, cardiac arrhythmias and weight gain. These properties make tricyclic antidepressants not favorable for treatment of elderly patients and, in particular, patients with cardiovascular diseases. The therapeutic effects of known antidepressants are revealed only several weeks after beginning their application to a patient.

The role of serotonin in the treatment of depressive and anxiety disorders is underscored by the therapeutic action of selective 5-HT reuptake inhibitors acting to enhance the degree of activation of various 5-HT receptor subtypes [Blier P. and Ward M., Biol. Psychiatry, 2003, 53, 193-203]. Another kind of antidepressants was discovered in the last thirty years based on serotonin reuptake inhibition. Such class of medicines named selective serotonin reuptake inhibitors (SSRIs) has had significant success in treating depression and related disorders and has become among the most prescribed drugs since 1980s. But, the use of SSRIs leads to the indiscriminate activation of all serotonin receptors and as a result there may be undesirable actions of serotonin in undesirable pathways at undesirable receptor subtypes [Stahl S M., 2008, Essential Psychopharmacology (3^{rd} ed.), 531]. Moreover SSRIs also produce numerous side effects. For example, SSRIs including fluoxetine (Prozac), paroxetine, fluvoxamine, sertraline, and citalopram (Cipramil) are associated with gastrointestinal distress, jitteriness, agitation and sleep disruption.

It was shown that SSRIs e.g. fluoxetine (Prozac) could increase suicide probability in the first months of therapy especially in the case of children and teenage patients. Often the increased risk of suicide is connected with a fast stimulating action which occurs prior to any true antidepressant effect. As a result, suicide-prone patients may develop enough strength or power to realize their suicidal intention while still being in a saddened or labile mood.

Moreover many known antidepressants can induce anxiety, sleeping abnormalities or psychotic state which also can increase suicide risk. A lot of suicide and aggressive behavior cases have been reported in the United States occurred during the time a patient was on fluoxetine (Prozac) therapy.

Although there are various treatment approaches for depressive and anxiety disorders, known antidepressant drugs have many disadvantages like a wide variety of responses, possible resistance to therapy, large latency period prior to desirable therapeutic effect and also a number of adverse effects, especially during long-term therapy. So despite many potent medicines being widely used in anti-depression therapy, a need still exists for new drugs with improved tolerability and adequate efficacy.

Promising way to search for novel antidepressant drugs is a study of new classes of chemical compounds considering the structures of known antidepressants.

Diaziridine derivatives are expected to act as potentially active antidepressant class since the fact some representatives of that have shown activity as monoaminooxydaze (MAO) inhibitors (C. J. Paget, C. S. Davis, J. Med. Chem., 1964 7, 626; R. G. Kostianovsky, G. V. Shustov, O. G. Nabiev, S. N. Denisenko, S. A. Sukhanova, E. F. Lavretskaya, Khim-Pharm. J., 1986, 20, 671).

### SUMMARY OF THE INVENTION

In one general aspect, the present invention provides compounds of formula (I), use of these compounds, their pharmaceutical acceptable salts, pharmaceutical compositions containing them, for treatment mental disorders, especially depressions of different etiology, and methods for their preparation. According to the present invention, compounds for treatment of mental disorders are meso-1S,2S,1′R,2′R-1-[ω-(3,3-dialkyldiaziridin-1-yl)alkyl]-3,3-dialkyldiaziridines having the following formula:

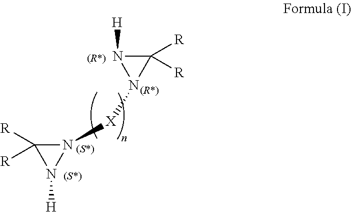

wherein n is an integer from 2 to 12,

R is selected from (C₁-C₁₀)alkyl, (C₂-C₁₀)alkenyl, (C₂-C₁₀)alkynyl, aryl, (C₃-C₁₀)cycloalkyl, (C₃-C₁₀)heterocycloalkyl, (C₁-C₁₀)alkoxy, amino, CO₂(C₁-C₁₀)alkyl, CO(C₁-C₁₀)alkyl(aryl), (C₁-C₁₀)alkylamino, CO(C₁-C₁₀)alkyl(heteroarylaryl) wherein R may be optionally substituted by 1 or more substituents selected from halo, hydroxy, oxy, cyano, aryl, aryloxy, heteroaryloxy, hetreroaryl, (C₁-C₁₀)alkyl, (C₂-C₁₀)alkenyl, (C₂-C₁₀)alkynyl, (C₃-C₁₀)cycloalkyl, (C₃-C₁₀)heterocycloalkyl, (C₁-C₁₀)alkoxy, amino, CO₂(C₁-C₁₀)alkyl, CO(C₁-C₁₀)alkyl(aryl), (C₁-C₁₀)alkylamino, or

two R substituents attached to the same carbon can be taken together with the carbon to which they are attached to form a cycle selected from (C₃-C₁₀)cycloalkylene, (C₃-C₁₀)heterocycloalkylene which can be optionally substituted by halo, hydroxy, cyano, (C₁-C₁₀)alkyl, (C₂-C₁₀)alkenyl, (C₂-C₁₀)alkynyl, (C₃-C₁₀)cycloalkyl, aryl,

X is CZ₂-Y, where Z is independently selected from H, halo, cyano, optionally substituted (C₁-C₁₀)alkyl, optionally substituted (C₂-C₁₀)alkenyl, optionally substituted (C₂-C₁₀)alkynyl, optionally substituted (C₃-C₁₀)cycloalkyl,

Y is selected from single bond, CZ₂, O, S, NH, N((C₁-C₁₀)alkyl),

or pharmaceutically acceptable salts thereof, solvates thereof such as, hydrates, and pharmaceutical compositions containing such compounds,

with the proviso that the compound of formula I is not meso-1S,2S,1′R,2′R-1-[2-(3,3-dimethyldiaziridin-1-yl)ethyl]-3,3-dimethyldiaziridine (R═Me, X═CH2, n=2).

A meso compound or meso isomer is a non-optically active member of a set of stereoisomers, at least two of which are optically active. This means that despite containing two or more stereocenters (chiral centers) compound is not chiral.

In another general aspect, the present invention relates to compound of formula (I′), namely meso-1S,2S,1′R,2′R-1-[2-(3,3-dimethyldiaziridin-1-yl)ethyl]-3,3-dimethyldiaziridine, use of this compound to treat mental disorders, especially depressions of different etiology, and methods for preparation of this compound. According to this aspect of the present invention, the compound for treatment of mental disorders is meso-1S,2S,1′R,2′R-1-[2-(3,3-dimethyldiaziridin-1-yl)ethyl]-3,3-dimethyldiaziridine having the following formula:

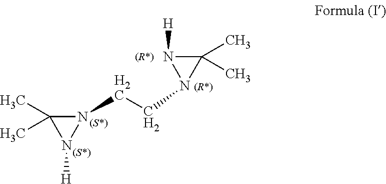

and salts thereof, solvates thereof such as hydrates and any crystal form (including co-crystals and polymorphs) thereof prepared by crystallization of any solvent and pharmaceutical compositions containing such compounds.

Compounds of formula I and formula I′ show high antidepressant activity comparable to known antidepressant drugs (Nyalamidum, Imipraminum, pyrazidolum) and develop their desirable therapeutic effect faster they were shown to be effective even after a single administration. They have a low acute and chronic toxicity compared to known antidepressants (LD₅₀=1000-1500 mg/kg, compared to LD₅₀ of Nyalamidum=820 mg/kg, of Imipraminum=150 mg/kg, of Prozac=340 mg/kg, of cypramile=1.7 mg/kg), do not produce pathological changes of any internal, hematological and biochemical parameters, and are therefore in long-term application.

The antidepressant action of compounds of formula I and formula I′ are connected mainly with serotonin- and dopamine-mimetic activities which is necessary for modulation of neurochemical processes of the brain. They have the original complex mechanism of action on the one hand binding with high affinity to the dopamine D-2-receptors of a striatum and modulating them and on the other hand inhibiting MAO activity in brain and liver tissues. Moreover it is worth noting they have no cholinolytic action.

The antidepressant activity can be accompanied with antipsychotic and tranquilizing activity. In a spectrum of psychotropic action, antineurotic action is present based on a showing by normalization of behavioral and biochemical deviations.

They render influence on processes, stimulating education of animals, execution of skill in poorly trained rats, and promoting conservation of an important memory.

They show adrenopositive activity ability to decease return seizure of noradrenalin in a cortex of the brain.

They have no cardiotoxic or hepatotoxic action, do not exert an influence on rhythm, frequency or strength of systole. They have no direct influence on myocardium, do not reduce conduction time, exert no negative inotropic effect, do not lower arterial pressure, or change any response to adrenaline. This property is very important as tricyclic antidepressants are known to induce orthostatic hypotension, tachycardia, reduction of P-Q, Q-R-S and Q-T interval, auricle and ventricle arrhythmia.

The nature of action of compounds I and I′ allows their use for treatment of patients resistant to therapy by the standard antidepressants, including elderly patients resistant to standard antidepressants.

This invention also relates to pharmaceutical compositions for treating psychiatric and depressive disorders. Examples of such mental conditions that can be treated with the compounds of formula I and I′ and pharmaceutical acceptable salts thereof are adjustment disorders, alcoholism, Alzheimer's disease, anorexia anxious depression, anxiety disorders, bipolar disorder, bulimia, cannabis compound abuse, childhood disorders, cognitive disorders, depression of different origin (e.g. melancholic depression, severe depression, psychotic depression) depressive disease, dissociative disorders, dysthymia eating disorders, factitious disorders, feeling of reluctance, hypnotic disorder, impulse-control disorders, insomnia, gender identity disorders, labile or saddened mood, major depressive disorder, melancholia, menopause associated depression, mood disorders, obsessive-compulsive disorder, opioid abuse panic disorder, personality disorders, phobic disorders, posttraumatic stress disorder, premenstrual associated depression, primary hypersomnia, primary insomnia, psychosis, psychosis of different origin (e.g. alcohol psychosis, circular psychosis, involutional psychosis, psychosis associated with dementia, psychosis associated with Alzheimer's disease, psychosis associated with an organic brain syndrome, drug-induced psychosis), psychotic disorders, refractory depression, resistant depression, schizoaffective disorder, schizophrenia, sexual disorders, sleep disorders, somatoform disorders, schizophrenia, substance disorders, suicide intention, suicide, unipolar depression, and unmotivation.

Depressive disorders encompass the diagnoses of major depression, dysthymia, and atypical depression or depression not otherwise specified (“minor depression”). The different subgroups of depressive disorders are categorized and defined by the Diagnostic and Statistical Manual of Mental Disorders, Fourth Edition (DSM-IV). (American Psychiatric Association. Diagnostic and Statistical Manual of Mental Disorders, 4^{th} Ed., Primary Care Version (DSM-IV-PC). American Psychiatric Association Press, Washington, D.C. 1995). According to the DSM-IV, a diagnosis of “major depression” requires that a patient is showing at least five of the following nine symptoms during the diagnostic period: 1) depressed mood most of the day (most acute in the morning); 2) markedly diminished interest or pleasure in nearly all activities (anhedonia); 3) significant weight loss or gain; 4) insomnia or hypersomnia; 5) psychomotor agitation or retardation; 6) fatigue or energy loss; 7) feelings of guilt and worthlessness; 8) impaired concentration and indecisiveness; and 9) recurring thoughts of death or suicide. To support a diagnosis of major depression, a depressed mood or loss of interest (anhedonia) must be one of the five observed symptoms. In contrast, a diagnosis of “atypical depression” or “depression not otherwise specified” (also referred to as “minor depression”), the most common form of depression, requires between 2 and 4 depressive symptoms be present daily or for most of the day for at least a two week period. Dysthymia is a chronic, low intensity mood disorder characterized by anhedonia, low self esteem and low energy that persists for more than two years, consecutively. Seasonal affective disorder is considered to be a form of major depression characterized by seasonal variation.

The present invention also relates to methods for the preparation of meso-form of compounds of formula I and I′.

The present invention also relates to pharmaceutical compositions that consist of or consist essentially of a compound of Formula I as the active ingredient without the presence of the compound of Formula I′. The pharmaceutical compositions include a compound of Formula I and one or more pharmaceutically acceptable excipients but does not include the compound of Formula I′.

### DETAILED DESCRIPTION OF THE INVENTION

Synthesis of compounds according to formula I and I′ as the meso-form is based on an ability of the diaziridine ring to epimerize at heating. During synthesis the compounds according to formula I and I′ are formed as a mixture of meso form and racemate in ratio 1:1. The formed meso-form that displays antidepressive activity is isolated by crystallization from acetone without stirring followed by filtration. Then acetone is evaporated and the remainder refluxed in CHCl₃ for 3 hours. This solution contains the mixture of meso-form and racemate in a ratio of 1:1. The meso-form is isolated and the rest is converted once more into a mixture of the meso-form and the racemate. The above-mentioned procedure is repeated until all of the racemate is converted into the meso-form (typically, 4-5 times). The last 2 steps are best carried out with an accumulation of the portion remaining after isolation of the meso-form resulting after several experiments.

There are three methods for the synthesis of 1-[ω-(3,3-dialkyldiaziridin-1-yl)alkyl]-3,3-dialkyldiaziridines (compounds I) as the mixture meso-form and racemate. The first method is based on a reaction of ketoxime arylsulfonates 2a,b with α,ω-diamines 3 in aprotic solvents in the presence of secondary or tertiary amines (scheme 1).

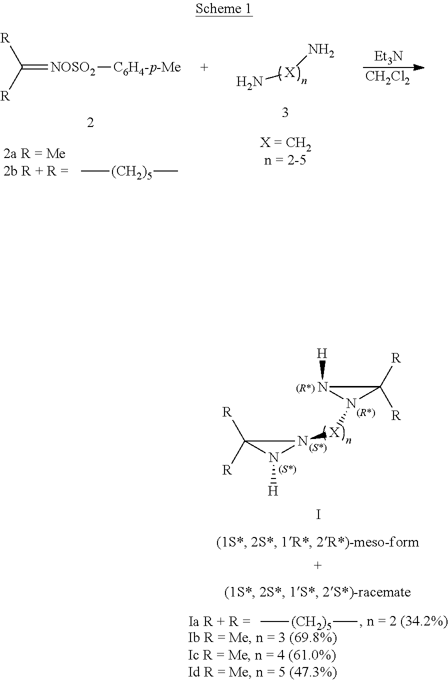

The second method is based on a reaction of ketones, α,ω-diamines 3 and hydroxylamine-)-O-sulfonic acid (HASA) in a mixture of methanol-water at pHₒₚₜ (scheme 2).

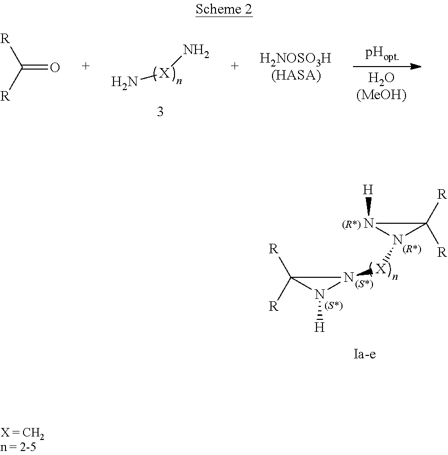

pHₒₚₜ is used to refer to the optimum pH for obtaining the final diaziridines of formula I with the highest yields. It was found that the highest yield of diaziridines 1 in water was achieved at an optimum pH (pHₒₚₜ) that shifts to a less alkaline region as the —I-effect of the substituents in the carbonyl compound increases and the pKBH+ value of the amine decreases. Further explanation can be found, for example, in the following references: (a) Kuznetsov, V. V.; Makhova, N. N.; Strelenko, Yu. A.; Khmel'nitskii, L. I. Izv. Acad. Nauk, SSSR, Ser. Khim. 1991, 2861 [Bull. Acad. Sci. USSR, Div. Chem. Sci. 1991, 40, 2496 (Engl. Transl.)] and (b) Kuznetsov, V. V.; Makhova, N. N.; Khmel'nitskii, L. I. Izv. Acad. Nauk, Ser. Khim. 1997, 1410 [Russ. Chem. Bull. 1997, 46, 1354 (Engl. Transl.)]. The contents of both of these journal articles are incorporated herein by reference in their entirety and, in particular, for their disclosure of the optimized pH and techniques for achieving the optimum pH.

The third method is based on a reaction of ketones, 1-(ω-aminoalkyl)-3,3-dialkyldiaziridines 4 and HASA in the mixture methanol-water at pHₒₚₜ (scheme 3).

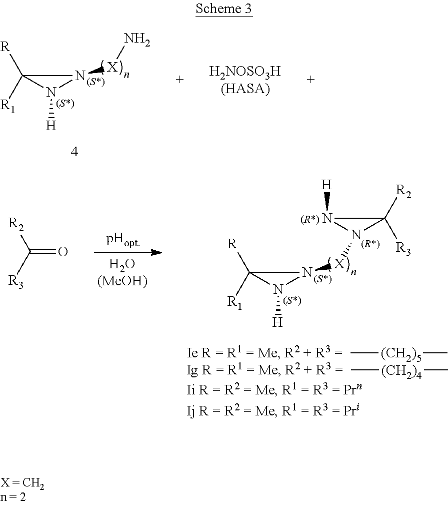

The initial 1-(ω-aminoalkyl)-3,3-dialkyldiaziridines 4 were synthesized based on a reaction of corresponding ketones, HASA and wacylaminoalkylamine 5 through intermediates 6 followed by their basic hydrolysis (scheme 4).

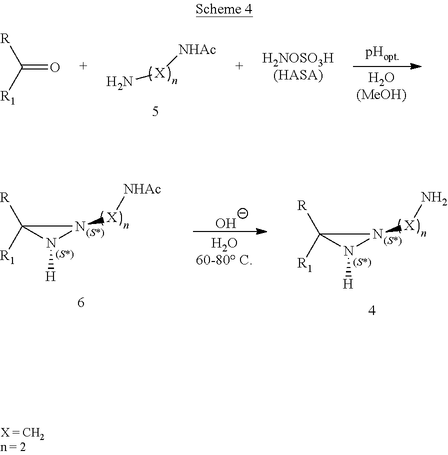

All compounds I obtained by the above-mentioned methods are the mixture of the meso-form and the racemate in a ratio of 1:1. Since only the meso-form can be used for the treatment of mental disorders, all racemates were converted into meso-forms. This process is based on the capacity of diaziridine ring to epimerize. The formed meso-forms of compounds I were isolated by crystallization from acetone without stirring followed by filtration. Then acetone is evaporated and the remainder is refluxed in CHCl₃ for 3 hours. This solution again contained the mixture of meso-form and racemate in a ratio of 1:1. Meso-form is isolated and the rest is once more converted into the mixture of meso-form and racemate. The above-mentioned procedure is repeated until all racemate is converted into meso-form (4-5 times). The last 2 steps are recommended to be carried out with the material resulting from the isolation step after several experiments.

Another way to separate diastereomeres is to apply HPLC, or flash chromatography.

The following examples of the preparation of compounds of the formula I illustrate this invention. These examples of preferred compounds of formula I include but are not limited to:

1. meso-1S,2S,1′R,2′R-1-[2-(1,2-diazaspiro[2.5]oct-1-yl)ethyl]-1,2-diazaspiro[2.5]octane (Ia)
2. meso-1S,2S,1′R,2′R-1-[3-(3,3-dimethyldiaziridin-1-yl)propyl]-3,3-dimethyldiaziridine (Ib)
3. meso-1S,2S,1′R,2′R-1-[4-(3,3-dimethyldiaziridin-1-yl)butyl]-3,3-dimethyldiaziridine (Ic)
4. meso-1S,2S,1′R,2′R-1-[5-(3,3-dimethyldiaziridin-1-yl)pentyl]-3,3-dimethyldiaziridine (Id)
5. meso-1S,2S,1′R,2′R-1-[2-(3,3-dimethyldiaziridin-1-yl)ethyl]-1,2-diazaspiro[2.5]octane (Ie)
6. meso-1S,2S,1′R,2′R-1-[2-(1,2-diazaspiro[2.4]hept-1-yl)ethyl]-1,2-diazaspiro[2.4]heptane (If)
7. meso-1S,2S,1′R,2′R-1-[2-(3,3-dimethyldiaziridin-1-yl)ethyl]-1,2-diazaspiro[2.4]heptane (Ig)
8. meso-1S,2S,1′R,2′R-1-[2-(1,2-diazaspiro[2.4]hept-1-yl)ethyl]-1,2-diazaspiro[2.5]octane (Ih)
9. meso-1S,2S,1′R,2′R-1-[2-(3-methyl-3-propyldiaziridin-1-yl)ethyl]-3-methyl-3-propyldiaziridine (Ii)
10. meso-1S,2S,1′R,2′R-1-[2-(3-isopropyl-3-methyl-diaziridin-1-yl)ethyl]-3-methyl-3-isopropyldiaziridine (Ij)

The following examples of the preparation of compounds of the formula I illustrate this invention. These examples of preferred compounds of formula I include but are not limited to:

11. meso-1S ,2S ,1′R,2′R-1-[2-(3,3-diethyldiaziridin-1-yl)ethyl]-3,3-diethyldiaziridine (IIa)
12. meso-1S ,2S ,1′R,2′R-1-[2-(3,3-dipropyldiaziridin-1- yl)ethyl]-3,3-dipropyldiaziridine (IIb)
13. meso-1S ,2S ,1′R,2′R-1-[2-(3,3-dibutyldiaziridin-1-yl)ethyl]-3,3-dibutyldiaziridine (IIc)
14. meso-1S ,2S ,1′R,2′R-1-[2-(3,3-dipentyldiaziridin-1-yl)ethyl]-3,3-dipentyldiaziridine (IId)
15. meso-1S ,2S ,1′R,2′R-1-[2-(3,3-dihexyldiaziridin-1-yl)ethyl]-3,3-dihexyldiaziridine (IIe)
16. meso-1S ,2S ,1′R,2′R-1-[2-(3,3-diheptyldiaziridin-1-yl)ethyl]-3,3-diheptyldiaziridine (IIf)
17. meso-1S ,2S ,1′R,2′R-1-[2-(3,3-dioctyldiaziridin-1-yl)ethyl]-3,3-dioctyldiaziridine (IIg)
18. meso-1S ,2S ,1′R,2′R-1-[2-(3,3-dinonyldiaziridin-1-yl)ethyl]-3,3-dinonyldiaziridine (IIh)
19. meso-1S ,2S ,1′R,2′R-1-[2-(3,3-didecyldiaziridin-1-yl)ethyl]-3,3-didecyldiaziridine (IIi)
20. meso-1S ,2S ,1′R,2′R-1-[2-(3-methyl-3-ethyldiaziridin-1-yl)ethyl]-3-methyl-3-ethyldiaziridine (IIj)

The following examples of the preparation of compounds of the formula I illustrate this invention. These examples of preferred compounds of formula I include but are not limited to:

21. meso-1S,2S,1′R,2′R-1-[3-(3,3-diethyldiaziridin-1-yl)propyl]-3,3-diethyldiaziridine (IIIa)
22. meso-1S,2S,1′R,2′R-1-[3-(3,3-dipropyldiaziridin-1-yl)propyl]-3,3-dipropyldiaziridine (IIIb)
23. meso-1S,2S,1′R,2′R-1-[3-(3,3-dibutyldiaziridin-1-yl)propyl]-3,3-dibutyldiaziridine (IIIc)
24. meso-1S,2S,1′R,2′R-1-[3-(3,3-dipentyldiaziridin-1-yl)propyl]-3,3-dipentyldiaziridine (IIId)
25. meso-1S,2S,1′R,2′R-1-[3-(3,3-dihexyldiaziridin-1-yl)propyl]-3,3-dihexyldiaziridine (IIIe)
26. meso-1S,2S,1′R,2′R-1-[3-(3,3-diheptyldiaziridin-1-yl)propyl]-3,3-diheptyldiaziridine (IIIf)
27. meso-1S,2S,1′R,2′R-1-[3-(3,3-dioctyldiaziridin-1-yl)propyl]-3,3-dioctyldiaziridine (IIIg)
28. meso-1S,2S,1′R,2′R-1-[3-(3,3-dinonyldiaziridin-1-yl)propyl]-3,3-dinonyldiaziridine (IIIh)
29. meso-1S,2S,1′R,2′R-1-[3-(3,3-didecyldiaziridin-1-yl)propyl]-3,3-didecyldiaziridine (IIIi)
30. meso-1S,2S,1′R,2′R-1-[3-(3-methyl-3-ethyldiaziridin-1-yl)propyl]-3-methyl-3-ethyldiaziridine (IIIj)

The following examples of the preparation of compounds of the formula I illustrate this invention. These examples of preferred compounds of formula I include but are not limited to:

31. meso-1S,2S,1′R,2′R-1-[4-(3,3-diethyldiaziridin-1-yl)butyl]-3,3-diethyldiaziridine (IVa)
32. meso-1S,2S,1′R,2′R-1-[4-(3,3-diprop yldiaziridin-1-yl)butyl]-3,3-dipropyldiaziridine (IVb)
33. meso-1S,2S,1′R,2′R-1-[4-(3,3-dibutyldiaziridin-1-yl)butyl]-3,3-dibutyldiaziridine (IVc)
34. meso-1S,2S,1′R,2′R-1-[4-(3,3-dipentyldiaziridin-1-yl)butyl]-3,3-dipentyldiaziridine (IVd)
35. meso-1S,2S,1′R,2′R-1-[4-(3,3-dihexyldiaziridin-1-yl)butyl]-3,3-dihexyldiaziridine (IVe)
36. meso-1S,2S,1′R,2′R-1-[4-(3,3-diheptyldiaziridin-1-yl)butyl]-3,3-diheptyldiaziridine (IVf)
37. meso-1S,2S,1′R,2′R-1-[4-(3,3-dioctyldiaziridin-1-yl)butyl]-3,3-dioctyldiaziridine (IVg)
38. meso-1S,2S,1′R,2′R-1-[4-(3,3-dinonyldiaziridin-1-yl)butyl]-3,3-dinonyldiaziridine (IVh)
39. meso-1S,2S,1′R,2′R-1-[4-(3,3-didecyldiaziridin-1-yl)butyl]-3,3-didecyldiaziridine (IVi)
40. meso-1S,2S,1′R,2′R-1-[4-(3-methyl-3-ethyldiaziridin-1-yl)butyl]-3-methyl-3-ethyldiaziridine (IVj)

Similarly, there are three methods for the synthesis of 1-[ω-(3,3-dialkyldiaziridin-1-yl)alkyl]-3,3-dialkyldiaziridines (formula I′) as the mixture meso-form and racemate. The first method is based on a reaction of ketoxime arylsulfonates 2 with α,ω-diamines 3 in aprotic solvents in the presence of secondary or tertiary amines (scheme 1a).

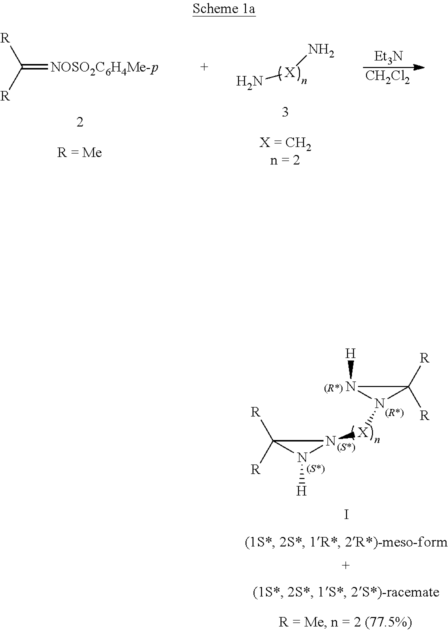

The second method is based on a reaction of ketones, α,ω-diamines 3 and hydroxylamine-O-sulfonic acid (HASA) in a mixture of methanol-water at pHₒₚₜ (scheme 2a).

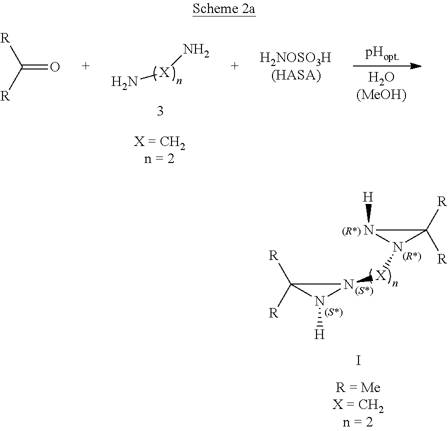

pHₒₚₜ is used to refer to the optimum pH for obtaining the final diaziridines of formula I with the highest yields. It was found that the highest yield of diaziridines 1 in water was achieved at an optimum pH (pHₒₚₜ) that shifts to a less alkaline region as the —I-effect of the substituents in the carbonyl compound increases and the pKBH+ value of the amine decreases. Further explanation can be found, for example, in the following references: (a) Kuznetsov, V. V.; Makhova, N. N.; Strelenko, Yu. A.; Khmel'nitskii, L. I. Izv. Acad. Nauk, SSSR, Ser. Khim. 1991, 2861 [Bull. Acad. Sci. USSR, Div. Chem. Sci. 1991, 40, 2496 (Engl. Transl.)] and (b) Kuznetsov, V. V.; Makhova, N. N.; Khmel'nitskii, L. I. Izv. Acad. Nauk, Ser. Khim. 1997, 1410 [Russ. Chem. Bull. 1997, 46, 1354 (Engl. Transl.)]. The contents of both of these journal articles are incorporated herein by reference in their entirety and, in particular, for their disclosure of the optimized pH and techniques for achieving the optimum pH.

The third method is based on a reaction of ketones, 1-(ω-aminoalkyl)-3,3-dialkyldiaziridines 4 and HASA in the mixture methanol-water at pHₒₚₜ (scheme 3a).

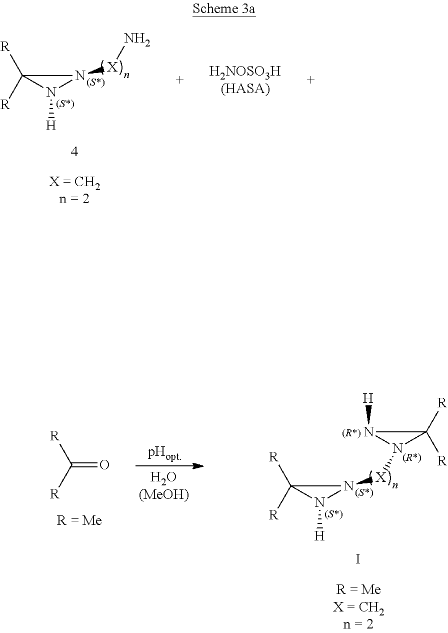

The initial 1-(ω-aminoalkyl)-3,3-dialkyldiaziridines 4 were synthesized based on a reaction of corresponding ketones, HASA and wacylaminoalkylamine 5 through intermediates 6 followed by their basic hydrolysis (scheme 4a).

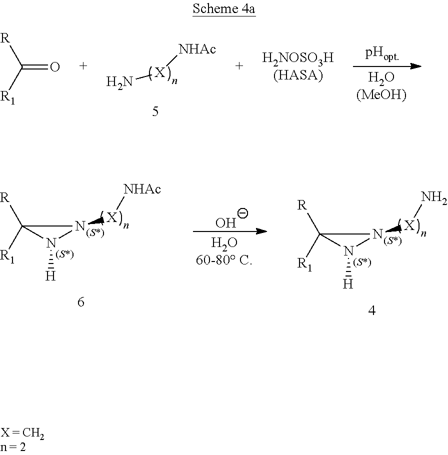

The compound I′ obtained by the above-mentioned methods is the mixture of the meso-form and the racemate in a ratio of 1:1. Since only the meso-form can be used for the treatment of mental disorders, racemate was converted into meso-forms. This process is based on the capacity of diaziridine ring to epimerize. The formed meso-form of compound I were isolated by crystallization from acetone without stirring followed by filtration. Then acetone is evaporated and the remainder is refluxed in CHCl₃ for 3 hours. This solution again contained the mixture of meso-form and racemate in a ratio of 1:1. Meso-form is isolated and the rest is once more converted into the mixture of meso-form and racemate. The above-mentioned procedure is repeated until all racemate is converted into meso-form (4-5 times). The last 2 steps are recommended to be carried out with the material resulting from the isolation step after several experiments.

Another way to separate diastereomeres is to apply HPLC or flash chromatography.

The compounds of the formula I and I′ have a basic nature and are capable of forming a wide variety of different salts with various inorganic and organic acids. The acids that can be used to prepare the pharmaceutically acceptable salts are those which form nontoxic salts, e.g. salts containing pharmaceutically acceptable anions, such as phosphates, acetates, oxalates, succinates, maleates, benzoates, etc.

The compounds of the formula I and I′ and their pharmaceutically acceptable salts are useful for the treatment of adjustment disorders, alcoholism, Alzheimer's disease, anorexia anxious depression, anxiety disorders, bipolar disorder, bulimia, cannabis compound abuse, childhood disorders, cognitive disorders, depression of different origin (e.g. melancholic depression, severe depression, psychotic depression) depressive disease, dissociative disorders, dysthymia eating disorders, factitious disorders, feeling of reluctance, hypnotic disorder, impulse-control disorders, insomnia, gender identity disorders, labile or saddened mood, major depressive disorder, melancholia, menopause associated depression, mood disorders, obsessive-compulsive disorder, opioid abuse panic disorder, personality disorders, phobic disorders, posttraumatic stress disorder, premenstrual associated depression, primary hypersomnia, primary insomnia, psychosis, psychosis of different origin (e.g. alcohol psychosis, circular psychosis, involutional psychosis, psychosis associated with dementia, psychosis associated with Alzheimer's disease, psychosis associated with an organic brain syndrome, drug-induced psychosis), psychotic disorders, refractory depression, resistant depression, schizoaffective disorder, schizophrenia, sexual disorders, sleep disorders, somatoform disorders, schizophrenia, substance disorders, suicide intention, suicide, unipolar depression, and lack of motivation.

Depressive disorders encompass the diagnoses of major depression, dysthymia, and atypical depression or depression not otherwise specified (“minor depression”). The different subgroups of depressive disorders are categorized and defined by the Diagnostic and Statistical Manual of Mental Disorders, Fourth Edition (DSM-IV). (American Psychiatric Association. Diagnostic and Statistical Manual of Mental Disorders, 4^{th} Ed., Primary Care Version (DSM-IV-PC). American Psychiatric Association Press, Washington, D.C. 1995). According to the DSM-IV, a diagnosis of “major depression” requires that a patient present with at least five of the following nine symptoms during the diagnostic period: 1) depressed mood most of the day (most acute in the morning); 2) markedly diminished interest or pleasure in nearly all activities (anhedonia); 3) significant weight loss or gain; 4) insomnia or hypersomnia; 5) psychomotor agitation or retardation; 6) fatigue or energy loss; 7) feelings of guilt and worthlessness; 8) impaired concentration and indecisiveness; and 9) recurring thoughts of death or suicide. To support a diagnosis of major depression, a depressed mood or loss of interest (anhedonia) must be one of the five observed symptoms. In contrast, a diagnosis of “atypical depression” or “depression not otherwise specified” (also referred to as “minor depression”), the most common form of depression, requires between 2 and 4 depressive symptoms be present daily or for most of the day for at least a two week period. Dysthymia is a chronic, low intensity mood disorder characterized by anhedonia, low self esteem and low energy that persists for more than two years, consecutively. Seasonal affective disorder is considered to be a form of major depression characterized by seasonal variation.

In addition the compounds of the formula I and I′ can be applied in catastrophe medicine which is recently became one of the most urgent medicine problems.

In implementation of antidepressive action of compounds of the formula I and I′ the most important features are serotonin- and dopamine-mimetic activities which are necessary for modulation of neurochemical processes of the brain. They have the original complex mechanism of action on the one hand binding with high affinity to the dopamine D-2-receptors of a striatum and modulating them as a result of course application and on the other hand inhibiting MAO activity in brain and liver tissues. Moreover it is worth noting they have no cholinolytic action.

The antidepressive activity can be accompanied by antipsychotic and tranquilizing activity. In a spectrum of psychotropic action antineurotic action is present showing by normalization of behavioral and biochemical deviations. They have no depressive activity.

They render influence on processes, stimulating education of animals, execution of skill in badly trained rats, and promote conservation of a memorable trace, e.g., important memory.

They show adrenopositive activity ability to decease return seizure of noradrenaline in a cortex of the brain.

They have no cardiotoxic and hepatotoxic action, nor influence rhythm, frequency and strength of systoli. They have no direct influence on myocardium, do not reduce conduction time, exert no negative inotropic effect, do not lower arterial pressure, nor change any response to adrenaline. This property is very important as tricyclic antidepressants are known to induce orthostatic hypotension, tachycardia, a reduction of P-Q, Q-R-S and Q-T interval, auricle and ventricle arrhythmia.

The nature of action of compounds I and I′ allows their use for treatment of patients resistant to therapy by the standard antidepressants, including elderly patients resistant to therapy with standard antidepressants.

Isomers may be separated using conventional techniques, e.g. chromatography or fractional crystallization. The diastereomers (meso-form and racemate) may be isolated by separation of isomer mixtures for instance by fractional crystallization, HPLC, or flash chromatography.

The compositions of the present invention may be formulated in a conventional manner using one or more pharmaceutically acceptable carriers. The active compounds of formula I and I′ may be formulated for oral, buccal, intransal, parenteral (e.g. intravenous, intramuscular or subcutaneous) or rectal.

In therapeutic use as agents for depression and anxiety the compounds of the present invention are used, alone or in combination with a pharmaceutically acceptable carrier or excipient. Standard pharmaceutical formulation techniques may be used, such as those disclosed in Remington's Pharmaceutical Sciences, Mack Publishing Company, Easton, Pa. (1990). The compounds of the present invention may be administered orally or parentally, neat or in combination with conventional pharmaceutical carriers. “Carrier” means one or more compatible substances that are suitable for administration to a mammal. Carrier includes solid or liquid fillers, diluents, hydrotopes, surface-active agents, and encapsulating substances. “Compatible” means that the components of the composition are capable of being commingled with the diaziridine compounds represented by structural formula (I) or I′, and with each other, in a manner such that there is no interaction which would substantially reduce the efficacy of the composition under ordinary use situations. Carriers must be of sufficiently high purity and sufficiently low toxicity to render them suitable for administration to the mammal being treated. The carrier can be inert, or it can possess pharmaceutical benefits, cosmetic benefits, or both.

The choice of carrier depends on the route by which the compounds represented by structural formula (I) and I′ will be administered and the form of the composition. The composition may be in a variety of forms, suitable, for example, for systemic administration (e.g., oral, rectal, nasal, sublingual, buccal, or parenteral).

The exact amounts of each component in the pharmaceutical composition depend on various factors. The amount of the diaziridine compound represented by structural formula (I) depends on the binding affinity (IC50) of the medicament selected. The amount of the carrier employed in conjunction with the medicament is sufficient to provide a practical quantity of material for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976), the entirety of each of which are incorporated herein in their entirety by reference for showing techniques and compositions of dosage forms.

Applicable solid carriers can include, without limitation, one or more substances which may also act as flavoring agents, lubricants, solubilizers, suspending agents, fillers, glidants, compression aids, binders, tablet-disintegrating agents, or encapsulating materials. In powders, the carrier may be a finely divided solid that may be in admixture with the finely divided active ingredient. In tablets, the active ingredient may be mixed with a carrier having suitable compression properties in suitable proportions and compacted in the shape and size desired. The powders and tablets may contain up to about 99% of the active ingredient.

Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes, and ion exchange resins. Liquid carriers may be used in preparing solutions, suspensions, emulsions, syrups, and elixirs.

The active ingredient of this invention may be dissolved or suspended in a pharmaceutically acceptable liquid carrier such as water, an organic solvent, a mixture of both, or pharmaceutically acceptable oils or fats. The liquid carrier may contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g., cellulose derivatives such as, without limitation, a sodium carboxymethyl cellulose solution), alcohols (including, without limitation, monohydric alcohols and polyhydric alcohols, e.g., glycols) and their derivatives, and oils (e.g., without limitation, fractionated coconut oil and arachis oil).

For parenteral administration the carrier can also be an oily ester such as ethyl oleate and isopropyl myristate. Sterile liquid carriers are used in sterile liquid form compositions for parenteral administration. Liquid pharmaceutical compositions that are sterile solutions or suspensions can be utilized by, for example, intramuscular, intraperitoneal, or subcutaneous injection. Sterile solutions can also be administered intravenously.

Oral administration may be either in liquid or solid composition form. In one embodiment, the pharmaceutical compositions containing the present compounds are in unit dosage form, e.g., as tablets or capsules. In such form, the composition may be sub-divided in unit dosages containing appropriate quantities of the active ingredients. The unit dosage forms can be packaged compositions, for example, packaged powders, vials, ampoules, prefilled syringes or sachets containing liquids. Alternatively, the unit dosage form can be, for example, a capsule or tablet itself, or it can be the appropriate number of any such compositions in package form. The therapeutically effective dosage to be used may be varied or adjusted by the physician and generally ranges from about 0.5 mg to about 750 mg, according to the specific condition(s) being treated and the size, age, and response pattern of the patient.

An effective amount of a compound according to the present invention will vary with the particular condition being treated, the age and physical condition of the patient being treated, the severity of the condition, the duration of treatment, the nature of concurrent therapy, the route of administration, the particular pharmaceutically-acceptable carrier utilized, and like factors within the knowledge and expertise of the attending physician. The compounds of the present invention may be administered to patients at a dosage of from about 0.7 to about 7000 mg per day, particularly about 1.0 to about 1000 mg. For example, for a normal human adult with a body weight of approximately 70 kg, the administration amount is translated into a daily dose of about 0.01 to about 100 mg per kg of body weight. The specific dosage employed, however, may vary depending upon the requirements of the patient, the severity of the patient's condition, and the activity of the compound. The determination of optimum dosages for a particular situation may be clinically determined and is within the level of skill of one or ordinary skill in the art. While these dosages are based upon a daily administration rate, the compounds of the present invention may also be administered at other intervals, such as twice per day, twice weekly, once weekly, or once a month. One of ordinary skill in the art would be able to calculate suitable effective amounts for other intervals of administration.

The exact amounts of each component in the pharmaceutical composition depend on various factors. The amount of the diaziridine compound added to the pharmaceutical composition is dependent on the IC50 of the compound, typically expressed in nanomolar (nM) units. For example, if the IC50 of the medicament is 1 nM, the amount of the diaziridine compound will be from about 0.001 to about 0.3%. If the IC50 of the medicament is 10 mM, the amount of the diaziridine compound will be from about 0.01 to about 1%. If the IC50 of the medicament is 100 nM, the amount of the diaziridine compound will be from about 0.1 to about 10%. If the IC50 of the medicament is 1000 nM, the amount of the diaziridine compound will be 1 to 100%, preferably 5% to 50%. If the amount of the diaziridine compound is outside the ranges specified above (i.e., lower), efficacy of the treatment may be reduced. One skilled in the art understands how to calculate and understand an IC50. The remainder of the composition, up to approximately 100%, may be a pharmaceutically acceptable carrier or excipient.

A better understanding of the present invention may be obtained in light of the following examples to illustrate, but are not to be construed to limit, the present invention. For oral administration, the pharmaceutical compositions may take the form of, for example, tablets or capsules prepared by conventional means with pharmaceutically acceptable excipients such a binding agents (e.g. polyvinylpyrrolidone or hydroxypropyl methylcellulose), lubricants (e.g. magnesium stearate, talk or silica). Tablets may be created by methods well known in the art using, e.g. acetylphtalylcellulose. Formulations for injection may be prepared in unit dosage form, e.g. in ampoules or in multi-dose containers, with an added preservative.

Specific examples of the anhydrous acid used for the preparation of the compound of formula (I) include, without limitation, hydrochloric acid, sulfuric acid, phosphoric acid, acetic acid, benzoic acid, citric acid, malonic acid, salicylic acid, malic acid, fumaric acid, oxalic acid, succinic acid, tartaric acid, lactic acid, gluconic acid, ascorbic acid, maleic acid, aspartic acid, benzene sulfonic acid, methane sulfonic acid, ethane sulfonic acid, hydroxymethane sulfonic acid, hydroxyethane sulfonic acid, and the like. For additional acids, one can refer to “Pharmaceutical Salts”, J. Pharm. Sci., 1977; 66(1): 1-19.

### EXAMPLES

The following Examples serve to further illustrate the present invention as applied to the compounds of Formula I and are not to be construed as limiting its scope in any way

1. meso-1S,2S,1′R,2′R-1-[2-(1,2-diazaspiro[2.5]oct-1-yl)ethyl]-1,2-diazaspiro[2.5]octane (Ia)
2. meso-1S,2S,1′R,2′R-1-[3-(3-(3,3-dimethyldiaziridin-1-yl)propyl]-3,3-dimethyldiaziridine (Ib)
3. meso-1S,2S,1′R,2′R-1-[4-(3,3-dimethyldiaziridin-1-yl)butyl]-3,3-dimethyldiaziridine (Ic)
4. meso-1S,2S,1′R,2′R-1-[5-(3,3-dimethyldiaziridin-1-yl)pentyl]-3,3-dimethyldiaziridine (Id)
5. meso-1S,2S,1′R,2′R-1-[2-(3,3-dimethyldiaziridin-1-yl)ethyl]-1,2-diazaspiro[2.5]octane (Ie)
6. meso-1S,2S,1′R,2′R-1-[2-(1,2-diazaspiro[2.4]hept-1-yl)ethyl]-1,2-diazaspiro[2.4]heptane (If)
7. meso-1S,2S,1′R,2′R-1-[2-(3,3-dimethyldiaziridin-1-yl)ethyl]-1,2-diazaspiro[2.4]heptane (Ig)
8. meso-1S,2S,1′R,2′R-1-[2-(1,2-diazaspiro[2.4]hept-1-yl)ethyl]-1,2-diazaspiro[2.5]octane (Ih)

### Experimental Part

Elemental analysis was performed by the CHN Analyzer Perkin-Elmer 2400. The IR spectra (v, cm-1) were measured using a SPECORD-M82 spectrometer. Mass spectra were measured using a Finnigan MAT INCOS-50 instrument. The NMR spectra were recorded using a Bruker AM-300 spectrometer at 300 MHz for 1H and 75 MHz for 13C Spectra in CDCl₃ as well as on Bruker AV-600 instrument with the frequencies 600.13, 150.90 and 60.81 MHz for ¹H, ¹³C and ¹⁵N, correspondingly. The chemical shifts of the signals of CDCl₃ of residual proton (7.27 ppm) and carbon (77.0 ppm) were used as the internal standard. The spectra were measured at 30° C. Analytical thin-layer chromatography (TLC) was conducted on silica gel plates (Silufol), eluent MeOH:NH₃ (25%)=10:1. Melting points were measured on a Gallenkamp instrument (Sanyo).

General method for the synthesis of meso -1-[ω-(3,3-dialkyldiaziridin-1-yl)alkyl]-3,3-dialkyldiaziridines I from ketoxime-O-sulfonates

α,ω-Diaminoalkane 3 (0.1 mol) and triethylamine (0.3 mol) were added to the solution of 0.2 mol ketoxime-O-sulfonate 2 in 40 ml of CH₂Cl₂ at temperature 10-15° C. The reaction mixture was stirred at 20-22° C. for 24 h and then at 25-30° C. for 90-120 h for full conversion of initial ester 2 (TLC-monitoring). Further 80 ml of CH₂Cl₂ and 30 g of fine pounded K₂CO₃ were added and stirring was continued for 5 h. The formed arylsulfonic acid potassium salt was filtered, washed with 50 ml of CH₂Cl₂ and solvent was evaporated on a rotary evaporator. The mixture stripped from the solvent was dissolved in acetone on heating and kept without stirring while cooling to room temperature. A precipitate was formed which was filtered, the solvent evaporated and the resulting product was dissolved in CHCl₃ and refluxed 5 h. Then CHCl₃ was evaporated and the resulting product was once more crystallized from acetone without stirring. This procedure was repeated 4-5 times to give the title compound, meso-1-[ω-(3,3-dialkyldiaziridin-1-yl)alkyl]-3,3-dialkyldiaziridine I.

For oily, not-crystallizing substances HPLC or flash chromatography can be used.

General method for the synthesis of meso-1-[ω-(3,3-dialkyldiaziridin-1-yl)alkyl]-3,3-dialkyldiaziridines I from ketones, α,ω-diaminoalkanes and hydroxylamine-O-sulfonic acid (HASA)

α,ω-Diaminoalkane (0.13 mol) was added dropwise to solution of ketone (0.6 mol) in 80 ml of water at −1-0° C., pH of solution was adjusted to 9 by addition of 50% aqueous H₂SO₄, at the same temperature HASA (0.5 mol) was added by portions keeping the pH at 8.5-9 by simultaneous addition of 40% aqueous KOH over 1.5 h. Then the reaction mixture was stirred for 2 h at 0° C. and pH 8.5-9, heated to 20° C. spontaneously, stirred 1.5-2 h at the same pH and temperature, the pH was raised to 12 and reaction mixture was kept for 12 h at this pH. Next day the reaction mixture was cooled to 0° C., pH was decreased to 7.3 by addition of 50% aqueous H₂SO₄, stirring was continued for 10-15 min, the precipitate K₂SO₄ was filtered and washed with CH₂Cl₂. The target product was extracted with CH₂Cl₂ until aqueous layer reacts positively to acidic solution of KI. The extracts were combined and dried with K₂CO₃ and the solvent was evaporated on a rotary evaporator at temperature ≦50° C. Then the meso-form of the title compound was obtained in a manner similar to general method described above.

General method for the synthesis of meso-1S,2S,1′R,2′R-1-[ω-(3,3-dialkyldiaziridin-1-yl)alkyl]-3,3-dialkyldiaziridines I from 1-(ω-aminoalkyl)-3,3-dialkyldiaziridine, ketones and hydroxylamine-O-sulfonic acid (HASA)

Ketone (0.1 mol) was added to a solution of 1-(ω-aminoalkyl)-3,3-dialkyldiaziridine (0.1 mol) in 100 ml of water at 0-5° C. and then 5-10 ml of MeOH was added if necessary until formation of a homogeneous solution. The pH of the solution was adjusted to 9 by addition of 50% aqueous H₂SO₄. HASA (0.1 mol, 11.3 g) was added by portions to the solution at 0-5° C. and pH 9-9.5 maintained constant by adding of 40% aqueous KOH over 1.5 h. Further reaction mixture was treated in a manner similar to general method described above.

1) meso-1S,2S,1′R,2′R - 1-[2-(1,2-diazaspiro[2.5]oct-1-yl)ethyl]-1,2-diazaspiro[2.5]octane (Ia) Yield 34.2%, m.p. 135-137° C. Found (%): C 67.21; H 10.46; N 22.42 C₁₄H₂₆N₄. Calculated: C 67.16; H 10.47; N 22.38. IR (v, cm⁻¹): 656, 828, 872, 1040, 1132, 1208, 1248, 1280, 1292, 1308, 1336, 1408, 1436, 1460, 2852, 2920, 2936, 2988, 3188. ¹H NMR (δ, ppm, CDCl₃): 1.25-1.75 (m, 20 H, —(CH₂)₅—); 1.85 (br.s., 2 H, NH); 2.65, 2.95 (both m, 4H, NCHₐ, ²J=−10.9 Hz, Δv=81.9 Hz, ³J=6.8 Hz,). ¹³C NMR (δ, ppm, CDCl₃): 24.83, 25.03, 25.48 (CH₂CH₂CH₂CH₂CH₂); 28.11, 38.85 (CH₂CH₂CH₂CH₂CH₂); 52.42 (NCH₂); 61.45 (C_{ring}). TLC: R_{f}0.67, eluent—MeOH:25% NH₃=10:1; mass: M⁺=250.
2) meso-1S,2S,1′R,2′R-1-[3-(3,3-dimethyldiaziridin-1-yl)prop-1-yl]-3,3-dimethyldiaziridine (Ib) Yield 69.7%; mass: M⁺=184.
3) meso-1S,2S,1′R,2′R-1-[4-(3,3-dimethyldiaziridin-1-yl)but-1-yl]-3,3-dimethyldiaziridine (Ic) Yield 61.0%, m.p. 82-83° C., mass: M⁺=198.
4) meso-1S,2S,1′R,2′R-1-[5-(3,3-dimethyldiaziridin-1-yl)pent-1-yl]-3,3-dimethyldiaziridine (Id) Yield 47.3%; mass: M⁺=212.
5) meso-1S,2S,1′R,2′R-1-[2-(3,3-dimethyldiaziridin-1-yl)ethyl]-1,2-diazaspiro[2.5]octane (Ie) Yield 55.0%; m.p. 101-103° C., mass: M⁺=210.
6) meso-1S,2S,1′R,2′R-1-[2-(1,2-diazaspiro[2.4]hept-1-yl)ethyl]-1,2-diazaspiro[2.4]heptane (If) Yield 47.0%; m.p. 127-128° C., mass: M⁺=222.
7) meso-1S,2S,1′R,2′R-1-[2-(3,3-dimethyldiaziridin-1-yl)ethyl]-1,2-diazaspiro[2.4]heptane (Ig) Yield 54.0%; m.p. 110-112° C., mass: M⁺=196
8) meso-1S,2S,1′R,2′R-1-[2-(1,2-diazaspiro[2.4]hept-1-yl)ethyl]-1,2-diazaspiro[2.5]octane (Ih) Yield 48.0%; m.p. 130-132° C., mass: M⁺=236.

The following example serves to further illustrate the present invention as applied to the compound of Formula la and are not to be construed as limiting its scope in any way: meso-1S ,2S ,1′R,2′R-1-[2-(3,3-dimethyldiaziridin-1-yl)ethyl]-3,3-dimethyldiaziridine (I′).

### Experimental Part

Elemental analysis was performed by the CHN Analyzer Perkin-Elmer 2400. The IR spectra (v, cm-1) were measured using a SPECORD-M82 spectrometer. Mass spectra were measured using a Finnigan MAT INCOS-50 instrument. The NMR spectra were recorded using a Bruker AM-300 spectrometer at 300 MHz for 1H and 75 MHz for 13C Spectra in CDCl₃ as well as on Bruker AV-600 instrument with the frequencies 600.13, 150.90 and 60.81 MHz for ¹H, ¹³C and ¹⁵N, correspondingly. The chemical shifts of the signals of CDCl₃ of residual proton (7.27 ppm) and carbon (77.0 ppm) were used as the internal standard. The spectra were measured at 30° C. Analytical thin-layer chromatography (TLC) was conducted on silica gel plates (Silufol), eluent MeOH:NH₃ (25%)=10:1. Melting points were measured on a Gallenkamp instrument (Sanyo).

General method for the synthesis of meso-1-[ω-(3,3-dialkyldiaziridin-1-yl)alkyl]-3,3-dialkyldiaziridines I from ketoxime-O-sulfonates

α,ω-Diaminoalkane 3 (0.1 mol) and triethylamine (0.3 mol) were added to the solution of 0.2 mol ketoxime-O-sulfonate 2 in 40 ml of CH₂Cl₂ at temperature 10-15° C. The reaction mixture was stirred at 20-22° C. for 24 h and then at 25-30° C. for 90-120 h for full conversion of initial ester 2 (TLC-monitoring). Further 80 ml of CH₂Cl₂ and 30 g of fine pounded K₂CO₃ were added and stirring was continued for 5 h. The formed arylsulfonic acid potassium salt was filtered, washed with 50 ml of CH₂Cl₂ and solvent was evaporated on a rotary evaporator. The mixture stripped from the solvent was dissolved in acetone on heating and kept without stirring while cooling to room temperature. A precipitate was formed which was filtered, the solvent was evaporated and the resulting product was dissolved in CHCl₃ and refluxed 5 h. Then CHCl₃ was evaporated and the resulting product was once more crystallized from acetone without stirring. This procedure was repeated 4-5 times to give the title compound, meso-1-[ω-(3,3-dialkyldiaziridin-1-yl)alkyl]-3,3-dialkyldiaziridine, Formula I′.

For oily not-crystallizing substances HPLC or flash chromatography can be used.

General method for the synthesis of meso-1-[ω-(3,3-dialkyldiaziridin-1-yl)alkyl]-3,3-dialkyldiaziridines I from ketones, ovo-diaminoalkanes and hydroxylamine-O-sulfonic acid (HASA)

α,ω-Diaminoalkane (0.13 mol) was added dropwise to solution of ketone (0.6 mol) in 80 ml of water at −1-0° C., pH of solution was adjusted to 9 by addition of 50% aqueous H₂SO₄, at the same temperature HASA (0.5 mol) was added by portions keeping the pH at 8.5-9 by simultaneous addition of 40% aqueous KOH over 1.5 h. Then the reaction mixture was stirred for 2 h at 0° C. and pH 8.5-9, heated to 20° C. spontaneously, stirred 1.5-2 h at the same pH and temperature, then pH was raised to 12 and reaction mixture was kept for 12 h at this pH. Next day the reaction mixture was cooled to 0° C., pH was decreased to 7.3 by addition of 50% aqueous H₂SO₄, stirring was continued for 10-15 min, the precipitate K₂SO₄ was filtered and washed with CH₂Cl₂. The target product was extracted with CH₂Cl₂ until aqueous layer doesn't react positively to acidic solution of KI. The extracts were combined and dried with K₂CO₃ and the solvent was evaporated on a rotary evaporator at temperature ≦50° C. Then the meso-form of the title compound was obtained in a manner similar to general method described above.

General method for the synthesis of meso-1S,2S,1′R,2′R-1-[ω-(3,3-dialkyldiaziridin-1-yl)alkyl]-3,3-dialkyldiaziridines I from 1-(ω-aminoalkyl)-3,3-dialkyldiaziridine, ketones and hydroxylamine-O-sulfonic acid (HASA)

Ketone (0.1 mol) was added to a solution of 1-(ω-aminoalkyl)-3,3-dialkyldiaziridine (0.1 mol) in 100 ml of water at 0-5° C. and then 5-10 ml of MeOH was added if necessary until formation of a homogeneous solution. The pH of the solution was adjusted to 9 by addition of 50% aqueous H₂SO₄. HASA (0.1 mol, 11.3 g) was added by portions to the solution at 0-5° C. and pH 9-9.5 maintained constant by adding of 40% aqueous KOH over 1.5 h. Further reaction mixture was treated in a manner similar to general method described above.

meso-1S,2S,1′R,2′R-1-[2-(3,3-dimethyldiaziridin-1-yl)ethyl]-3,3-dimethyldiaziridine (I′). Yield of mixture of diasteomers 77.5-87.5%. The final yield of meso-form of 1S,2S,1′R,2′R-1-[2-(3,3-dimethyldiaziridin-1-yl)ethyl]-3,3-dimethyldiaziridine (I′) was 65-75%. The structure was supported by elemental analysis, spectral characteristics and X-ray diffraction study (FIG. 1).

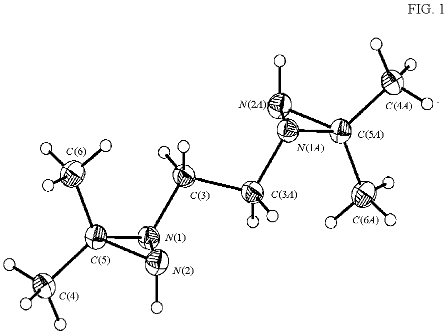

Found: C 56.61; H 10.73; N 32.58. C₈H₁₈N₄. Calculated: C 56.44; H 10.66; N 32.91. IR-(v, cm⁻¹): 656, 760, 808, 828, 976, 1004, 1072, 1104, 1128, 1212, 1276, 1300, 1320, 1352, 1384, 1448, 1464, 2864, 2932, 2964, 2996, 3008, 3212. ¹H NMR (δ, ppm, CDCl₃): 1.25. 1.28 (both s. 12 H, Me), 1,79 (br. s., 2H, NH), 2.52, 2,73 (both m. 4H, NCH₂, ²J=42.30 Hz, ³J=6.14 Hz, ³J=7.80 Hz). ¹³C NMR (δ, ppm, CDCl₃): 17.35 (Me); 28.16 (Me); 53.21 (NCH₂); 56.90 (C_{ring}). TLC: R_{f}0.59, eluent MeOH:25% NH₃=10:1. mass: M⁺=170.

The following biological examples are illustrative to demonstrate the potential usefulness of compounds of formula (I) and (I′) for the prevention and treatment of symptoms of depressive disorders and but not limiting the invention.

### Biological Examples

### Example 1

Black and white box test. (Compound of Formula I)

The black and white test (also named light-dark test) is based on the conflict of natural tendencies of rodents to avoid lighted and open areas and to explore novel environments. Relative time spent in exploring each compartment indicates the anxiety level of the animal: Avoidance of the brightly lit area is considered reflecting “anxiety-like” behaviors. When treated with anxiolytic drugs, rodents spend more time in this area, an effect purportedly due to a decrease in anxiety.

The C57BL/6J mice were treated intraperitoneally with compound of the invention, then anxiolytic efficacy of compounds of formula I was assessed by estimating the number of entries to the light zone (see Table 1).

### Example 2

Learned helplessness test. (Compound of Formula I)

The learned helplessness test in mice is the well-known animal model to determine antidepressant efficacy of compounds. Basically when animals learned to be helpless are given antidepressant drugs, they unlearn helplessness and start exerting control over their environment.

The C57BL/6J mice were treated intraperitoneally with compounds of formula I intraperitoneally at dose corresponding to ⅓ or 1/13 of lethal dose, then antidepressant activity of compounds was assessed by estimating latency time as a period in which animal is not trying to escape from stress (see Table 1).

### Example 3

Toxicity test. (Compound of Formula I)

Toxicity to mammals was measured after intraperitoneal injections of compounds of formula I to C57BL/6J mice. Median lethal dose (LD50) was calculated as described previously (see table 1).

TABLE 1

| | Number of | Latency time, %* | | |
|---|---|---|---|---|
| Treated groups | entries, %* | 1/3 LD50 | 1/13 LD50 | LD50, mg/kg |
| Ia | 56** | 56** | 72** | 611 |
| Ic | 62** | 188** | 104 | 1000 |
| Ie | 75** | 62** | 74** | 1125 |
| If | 60** | 95 | 94 | 1150 |

*data are expressed as percent of control (non-treated animals) taken as 100%
**p < 0.05 compared to control (non-treated animals)

These results seems to be very promising suggesting potentials of compounds of Formula I for treating depression.

The following biological examples are illustrative to demonstrate the potential usefulness of the compound of formula I′ for the prevention and treatment of symptoms of depressive disorders but are not limiting to the invention.

### Example 4

Behavioral despair test. (Compound of Formula I′)

Behavioral despair was proposed as a model to test for antidepressant activity by Porsolt et al. It was suggested that mice or rats forced to swim in a restricted space from which they cannot escape are induced to a characteristic behavior of immobility. This behavior reflects a state of despair which can be reduced by several agents which are therapeutically effective in human depression.

The male white rats (250-280 g) were treated intraperitoneally according to procedure described below. The animals were divided into five groups of ten rats and treated as follows. The rats of first group were used as controls. They were injected intraperitoneally with distilled water 24 h, 3 h, 40 minutes before the experiment. Second group was treated with Compound I′ at a dose 100 mg/kg i.p. 24 h, 3 h, 40 minutes before the experiment. Third group was treated with Amitriptyline at a dose 10 mg/kg i.p. using the same regimen as in groups above. Fourth and fifth groups were treated with Fluoxetin at a doses 10, 20 mg/kg as powder or tablet, respectively using the same regimen as in groups above.

The antidepressant activity was assessed by estimating immobility time as a period in which animal remains immobile during swimming (see Table 2). Statistical data processing was performed using “BioStat” tool for Windows.

TABLE 2

| | Dose, mg/kg | Immobility time, s |
|---|---|---|
| Control (group 1) | | 445.05 ± 14.63 |
| Compound 1 (group 2) | 100 | 291.55 ± 43.94* |
| Amitriptyline (group 3) | 10 | 145.62 ± 45.39* |
| Fluoxetin (group 4) | 10 | 337.45 ± 36.69 |
| Fluoxetin (group 5) | 20 | 218.45 ± 38.87* |

*p < 0.05 compared to control

Therefore compound I′ at a dose 100 mg/kg i.p. when administered three times 24 h, 3 h, 40 min before experiment displays significant antidepressant activity. The activity of compound Ia exceeds that of fluoxetin at 10 mg/kg, and is inferior to amitriptyline (10 mg/kg) and fluoxetin (20 mg/kg).

### Example 5

Learned helplessness test. (Compound of Formula I′)

As explained above, the learned helplessness test in mice is the well-known animal model to determine antidepressant efficacy of compounds. Basically when animals learned to be helpless are given antidepressant drugs, they unlearn helplessness and start exerting control over their environment. Antidepressant activity of compounds was assessed by estimating latency time as a period in which animal is not trying to escape from stress (see Table 3).

The C57BL/6J mice were treated intraperitoneally with Compound I′ intraperitoneally at a dose of 90 mg/kg once daily for 2, 6 or 12 days.

TABLE 3

| | Non- | | Non- | | Non- | |
|---|---|---|---|---|---|---|
| | escaping | Latency | escaping | Latency | escaping | Latency |
| | animals % | time, s | animals, % | time, s | animals, % | time, s |
| Treated groups | 2 days course | | 6 days course | | 12 days course | |
| non-stressed | 17 | 10.1 ± 1.3 | 16 | 9.5 ± 0.9 | 17 | 8.8 ± 1.0 |
| animals | | | | | | |
| (control 1) | | | | | | |
| stressed animals | 63^{x} | 16.1 ± 1.6^{x} | 54^{x} | 15.4 ± 1.7^{x} | 56^{x} | 15.2 ± 1.5^{x} |
| (control 2) | | | | | | |
| Ia | 18** | 13.6 ± 1.1** | 12** | 7.5 ± 0.9** | 1.4** | 6.2 ± 0.5** |
| (90 mg/kg, i.p.) | | | | | | |
| pirazidole | 39 | 13.6 ± 1.1 | 18 | 10.0 ± 1.4* | 14* | 8.3 ± 0.7** |
| (10 mg/kg, i.p) | | | | | | |
| melipramine | 43 | 13.5 ± 1.8 | 29 | 11.8 ± 1.5 | 11* | 9.2 ± 1.1* |
| (7 mg/kg, i.p.) | | | | | | |

^{x}p < 0.05 compared to control 1 (non-stressed animals)
*p < 0.05, **p < 0.01 compared to control 2 (stressed animals)

The compound of the formula I′ showed very promising antidepressive action reducing latency time and percent of mice who were not able to escape stress action. Single doses of Ia and comparative drugs were not effective, which is in accordance with data that single-dose antidepressant administration could not be effective in this model showing adaptive changes of brain neurochemical systems of animal. Antidepressive effect of compound of formula la was observed 2 days after treatment which is earlier than for comparative drugs. After 12 days of treatment latency time and non-escaping percent was even lower than in the negative control group (group of animals which were not stressed) (see Table 3).

### Example 6

Modified behavioral despair test. (Compound of Formula I′)

Nomura et al. (1982) published a modification of the despair swim test in mice involving small water wheel set in a water tank. Mice placed on this apparatus turned the wheel vigorously but when they abandoned attempts to escape from the water, the wheel stopped turning. Antidepressant activity of compounds was assessed by counting the number of rotations of the water wheel (see Table 4).

The male white rats (250-280 g) were treated intraperitoneally according to procedure described below. The animals were divided into five groups of ten rats and treated as follows. The rats of first group were used as controls. They were injected intraperitoneally with distilled water 24 h, 3 h, 40 minutes before the experiment. Second group was treated with Compound Ia at a dose 100 mg/kg i.p. 24 h, 3 h, 40 minutes before the experiment. Third group was treated with Amitriptyline at a dose 10 mg/kg i.p. using the same regimen as in groups above. Fourth and fifth groups were treated with Fluoxetin at a doses 10, 20 mg/kg as powder or tablet, respectively using the same regimen as in groups above.

The antidepressant activity was assessed by estimating immobility time as a period in which animal remains immobile during swimming (see Table 4). Statistical data processing was performed using “BioStat” tool for Windows.

TABLE 4

| | Dose, mg/kg | Rotation number |
|---|---|---|
| Control (group 1) | | 104.72 ± 19.41 |
| Compound 1 (group 2) | 100 | 156.48 ± 26.79* |
| Amitriptyline (group 3) | 10 | 165.93 ± 35.41* |
| Fluoxetin (group 4) | 10 | 108.34 ± 53.96 |
| Fluoxetin (group 5) | 20 | 142.74 ± 29.85* |

*p < 0.05 compared to control

Therefore compound I′ at a dose 100 mg/kg i.p. when administered three times 24 h, 3 h, 40 min before experiment displays significant antidepressant activity. The activity of compound Ia exceeds that of fluoxetin at 10 mg/kg, but is comparable to amitriptyline (10 mg/kg) and fluoxetin (20 mg/kg).

These results seems to be very promising suggesting potentials of compound I′, namely meso-1S,2S,1′R,2′R-1-[2-(3,3-dimethyldiaziridin-1-yl)ethyl]-3,3-dimethyldiaziridine and salts thereof, solvates thereof such as hydrates and any crystal form (including co-crystals and polymorphs) thereof prepared by crystallization of any solvent and pharmaceutical compositions containing such compounds, for treating depression.

As noted above, the compounds described herein have a basic nature and, as such, may be subject to degradation in an acidic environment, such as is found in the stomach. To address this potential for degradation, the compounds may be administered in an enteric coated dosage form or enteric coated pellets in a capsule. Enteric pharmaceutical formulations are manufactured in such a way that the product passes unchanged through the stomach of the patient, and dissolves and releases the active ingredient quickly when it leaves the stomach and enters the small intestine. Such formations have long been used, and conventionally are in tablet or pellet form, where the active ingredient is in the inner part of the tablet or pellet and is enclosed in a film or envelope, the “enteric coating”, which is insoluble in acid environments, such as the stomach, but is soluble in near-neutral environments such as the small intestine.

The compound may be provided in the form of enteric coated pellet comprising a) a core consisting of the compound and a pharmaceutically acceptable excipient; b) an optional separating layer; c) an enteric layer comprising an enteric polymer and an optional pharmaceutically acceptable excipient; and d) an optional finishing layer.

The Core

A preferred core for the pellet is prepared by applying a compound-containing layer to an inert bead. Such inert beads are conventionally used in pharmaceutical science, and are readily purchased in all industrial countries. A suitable bead is one prepared from starch and sucrose, for use in confectionery as well as in pharmaceutical manufacturing. However, beads of any pharmaceutically acceptable excipient may be used, including, for example, microcrystalline cellulose, vegetable gums, waxes, and the like. The primary characteristic of the inert bead is to be inert, with regard both to the drug and the other excipients in the pellet and with regard to the patient who will ultimately ingest the pellet.

The size of the beads depends on the desired size of the pellet to be manufactured. In general, pellets can be as small as 0.1 mm, or as large as 2 mm. A suitable bead may be from about 0.3 to about 0.8 mm, in order to provide finished pellets in a desired size range of from about 0.5 to about 1.5 mm in diameter.

A convenient manner of coating the beads with duloxetine is the “powder coating” process where the beads are moistened with a sticky liquid or binder, duloxetine is added as a powder, and the mixture is dried. Such a process is regularly carried out in the practice of industrial pharmacy, and suitable equipment is in daily use.

Additional solids may be added to the layer with the compound. These solids may be added to facilitate the coating process as needed to aid flow, reduce static charge, aid bulk buildup and form a smooth surface. Inert substances such as talc, kaolin, and titanium dioxide, lubricants such as magnesium stearate, finely divided silicon dioxide, crospovidone, and lactose may be used. The amounts of such substances are in the range from about a few tenths of 1% of the product, up to about 20% of the product. Such solids should be of fine particle size, less than 50 microns, to produce a smooth surface.

The compound is made to adhere to the beads by spraying a pharmaceutical excipient which is sticky and adherent when it is wet, and dries to a strong, coherent film. Pharmaceutical scientists are aware of and conventionally use many such substances, most of them polymers. Preferred such polymers include hydroxypropylmethylcellulose, hydroxypropylcellulose and polyvinylpyrrolidone. Additional such substances include, for example, methylcellulose, carboxymethylcellulose, acacia and gelatin. The amount of the adhering excipient is in the range from about a few tenths of 1% to about 5% of the product, and depends in large part on the amount of compound to be adhered to the bead.

Separating Layer

The optional separating layer between the compound-containing core and the enteric layer is not required, but is a useful feature of the formulation if there is any adverse interactions between the compound and the enteric polymer. The other functions of the separating layer are to provide a smooth base for the application of the enteric layer, to prolong the pellet's resistance to acid conditions, and to improve stability by protecting the compound from light exposure.

The smoothing function of the separating layer is purely mechanical, the objective of which is to improve the coverage of the enteric layer and to avoid thin spots in it, caused by bumps and irregularities on the core. Accordingly, the more smooth and free of irregularities the core can be made, the less material is needed in the separating layer, and the need for the smoothing characteristic of the separating layer may be avoided entirely when the compound is of extremely fine particle size and the core is made as close as possible to truly spherical.

In some cases, the separating layer can also act as a diffusional barrier to migrating core or enteric layer components dissolved in product moisture. The separating layer can also be used as a light barrier by opacifying it with agents such as titanium dioxide, iron oxides and the like.

In general, the separating layer is composed of coherent or polymeric materials, and finely powdered solid excipients which constitute fillers. When a sugar is used in the separating layer, it is applied in the form of an aqueous solution and constitutes part of or the whole of the coherent material which sticks the separating layer together. In addition to or instead of the sugar, a polymeric material may also be used in the separating layer. For example, substances such as hydroxypropylmethylcellulose, polyvinylpyrrolidone, hydroxypropylcellulose and the like may be used in small amounts to increase the adherence and coherence of the separating layer.

It is further advisable to use a filler excipient in the separating layer to increase the smoothness and solidity of the layer. Substances such as finely powdered talc, silicon dioxide and the like are universally accepted as pharmaceutical excipients and may be added as is convenient in the circumstances to fill and smooth the separating layer.

The separating layer may be applied by spraying aqueous solutions of the sugar or polymeric material, and dusting in the filler as has been described in the preparation of the compound-containing layer. The smoothness and homogeneity of the separating layer can be improved, however, if the filler is thoroughly dispersed as a suspension in the solution of sugar and/or polymeric material, and the suspension is sprayed on the core and dried.

Enteric Layer

The enteric layer is comprised of an enteric polymer, which must be chosen for compatibility with the compound and to provide the desired pH-dependent release. Examples of enteric polymers include: (meth)acrylate copolymer, shellac, HPMCP (hydroxypropylmethylcellulose phthalate), CAP (cellulose acetate phthalate), HPMC-AS (hydroxypropylmethylcellulose acetate succinate), polyvinyl acetate phthalate, carboxymethylethylcellulose, co-polymerized methacrylic acid/methacrylic acid methyl esters such as, for instance, compounds known under the trade name Eudragit L 12.5 or Eudragit L 100 (Rohm Pharma), or similar compounds used to obtain enteric coatings. The enteric coating layer can optionally contain a pharmaceutically acceptable plasticizer such as, for instance, cetanol, triacetin, citric acid esters such as, for instance, those known under the trade name Citroflex (Pfizer), phthalic acid esters, dibutyl succinate or similar plasticizers. The amount of plasticizer is usually optimized for each enteric coating polymer(s) and is usually in the range of 1-20% of the enteric coating polymer(s). Dispersants such as talc, colorants and pigments may also be included into the enteric coating layer.

Finishing Layer

A finishing layer over the enteric layer is not necessary in every instance, but frequently improves the elegance of the product and its handling, storage and machinability and may provide further benefits as well. The simplest finishing layer is simply a small amount, less than about 1%, of an anti-static ingredient such as talc or silicon dioxide, simply dusted on the surface of the pellets. Another simple finishing layer is a small amount, about 1%, of a wax such as beeswax melted onto the circulating mass of pellets to further smooth the pellets, reduce static charge, prevent any tendency for pellets to stick together, and increase the hydrophobicity of the surface.

More complex finishing layers may constitute a final sprayed-on layer of ingredients. For example, a thin layer of polymeric material such as hydroxypropylmethylcellulose, polyvinylpyrrolidone and the like, in an amount such as from a few tenths of 1% up to about 3%, may be applied. The polymeric material may also carry a suspension of an opacifier, a bulking agent such as talc, or a coloring material, particularly an opaque finely divided color agent such as red or yellow iron oxide. Such a layer quickly dissolves away in the stomach, leaving the enteric layer to protect the compound, but provides an added measure of pharmaceutical elegance and protection from mechanical damage to the product.

The following formulation examples provide guidance in making a formulation of the disclosed compounds.

### Formulation Example 1

10 mg compound/capsule

| Ingredient | Amount (mg) | |
|---|---|---|
| Beads | | |
| Microcrystalline cellulose | 30.00 | mg |
| Drug Layer | | |
| Compound I or I′ | 11.23 | |
| Beta Lactose | 48.77 | |
| Cross-linked polyvinylpyrrolidone | 6.00 | |
| Hydroxypropylcellulose | 0.72 | |
| Talc | 8.00 | |
| Separating Layer | | |
| Hydroxypropylmethylcellulose | 1.20 | |
| Polyethylene glycol 6000 | 4.60 | |
| Talc | 13.90 | |
| Titanium dioxide | 1.20 | |
| Enteric Layer | | |
| HPMC-AS LF (Shin-Etsu) | 44.70 | |
| Triethyl citrate | 10.70 | |
| Talc | 13.40 | |
| Finishing Layer | | |
| White Beeswax | 1.90 | |
| Hydrated silicon dioxide | 0.40 | |
| Total Weight | 196.70 | |

The drug layer is to be added to the beads in a CF granulator at a batch size of 3.6 kg. The hydroxypropylcellulose is to be dissolved in a minimum amount of water, and the solution slowly sprayed onto the agitating batch of beads, while the compound, lactose and crospovidone, as a mixture is to be intermittently added at a rate such that it would be adhered to the beads without loss through dusting. When the drug layer is fully formed, the talc is to be added in the same manner, and the beads dried in an oven at 55° C. for 1.5 hours, and then classified between 20 and 42 mesh screens.

Nest, the separating layer is applied in a Wurster column (Uni-Glatt, Glatt Air Techniques, Inc., Ramsey, N.J.). The hydroxypropylmethylcellulose and the polyethylene glycol are to be dissolved in water, and the talc and titanium dioxide dispersed in the solution with a homogenizer. The resulting suspension is to be sprayed onto the classified beads in the Wurster column.

The enteric coating suspension is to be prepared by first dissolving the triethyl citrate in water, cooling the solution to 15° C., and preparing a 7% w/v suspension of the HPMCAS-LF in the cool solution. The HPMCAS-LF and talc are to be added slowly, taking care to avoid foaming or the formation of aggregates of polymer. Then the partially formed granules are to be added to a fluidized bed coating device, provided with a Wurster column. The batch is to be fluidized with air at 70°-80° C. and the enteric suspension sprayed into the batch and adjusting the spray rate and air flow to provide appropriate agitation and avoid agglomeration. When the addition is complete, air flow is to be continued for 30 minutes to dry the batch.

Finally, the finishing layer is to be created by adding the beeswax to the product in the fluidized bed at 60° C. After cooling, the hydrated silicon dioxide is to be added to the pellets and mixed in the Wurster column. The batch is then to be cooled and filled into number #3 gelatin capsules.

### Formulation Examples 2 and 3

Manufacture of tablet cores batch

| | Example 2 | Example 3 |
|---|---|---|
| | Amount | Amount |
| Ingredient | (grams) | (mg) |
| Compound I or I′ | 400 | 426 |
| Lactose anhydrous | 420 | 409 |
| Crosslinked | 100 | 100 |
| Polyvinylpyrrolidone | | |
| Sodium carbonate | 15 | — |
| anhydrous | | |
| Methyl cellulose | 12 | 12 |
| Distilled water | 200 | 200 |
| Magnesium stearate | 30 | 30 |

The powder mixture of the compound, lactose, polyvinylpyrrolidone, and sodium carbonate were homogenized and granulated by the solution of methyl cellulose and water. The wet mass was dried in a fluidized bed dryer using an inlet air temperature of +50° C. for 30 minutes. The dried mixture was then forced through a sieve with an aperture of 0.5 mm. After mixing with magnesium stearate the granulate was tableted on a tableting machine using 6 mm punches. The tablet weight was 100 mg.

Subcoating

The tablets of Example 2 is to be subcoated with approximately 10% by weight of hydroxypropyl methylcellulose from a water solution using a perforated coating pan apparatus. The tablets of Example 3 is to be subcoated using the dry coating technique.

A tablet granulate containing lactose anhydrous (4,000 grams), polyvinylpyrrolidone (PVP) (180 grams), ethanol 95% (420 grams) and magnesium stearate (42 grams) is to be prepared as follows: granulate the lactose with a solution of PVP in ethanol and dry, and the admix in the magnesium stearate.

Next the tablet granulate is to be dry coated around the tablet cores of Examples 2 and 3 using a Manesty Dry Cota tableting machine. The resulting tablet weight of the dry cotaed tablets of Example 2 will be approximately 475 mg with 20 mg of the compound.

Enteric Coating

The subcoated tablets obtained above are next to be enteric coated using the same coating solution: Hydroxypropyl methylcellulose phthalate (1,500 g); Cetyl alcohol (105 g), Methylene chloride (15,000 g), Isopropanol (15,000 g) and Distilled water (3,150 g). The coating is to be applied in a perforated coating pan apparatus. An approximate amount of one kg of coating solution is to be applied for each kg of tablets.

While several particular forms of the invention have been illustrated and described, it will be apparent that various modifications and combinations of the invention detailed in the text and drawings can be made without departing from the spirit and scope of the invention. For example, references to materials of construction, methods of construction, specific dimensions, shapes, utilities or applications are also not intended to be limiting in any manner and other materials and dimensions could be substituted and remain within the spirit and scope of the invention. For example, the compounds of the general formula (I) described herein can be obtained by one of ordinary skill in the art according to the methods described in the patent. The substituents or their protected derivatives may be parts of staring materials. The protecting groups of some of the substituents can be removed by methods known to those of ordinary skill in the art and readily available, for example, in conventional organic synthesis books and texts. Accordingly, it is not intended that the invention is limited, except as by the appended claims.

1. A compound of the formula I as meso-form 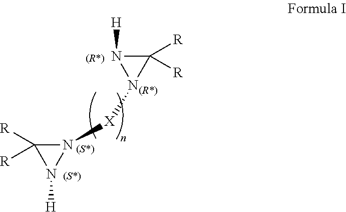
wherein n is an integer from 2 to 12,
R is selected from (C₁-C₁₀)alkyl, (C₂-C₁₀)alkenyl, (C₂-C₁₀)alkynyl, aryl, (C₃-C₁₀)cycloalkyl, (C₃-C₁₀)heterocycloalkyl, (C₁-C₁₀)alkoxy, amino, CO₂(C₁-C₁₀)alkyl, CO(C₁-C₁₀)alkyl(aryl), (C₁-C₁₀)alkylamino, CO(C₁-C₁₀)alkyl(heteroarylaryl), wherein R may be optionally substituted by 1 or more substituents selected from halo, hydroxy, oxy, cyano, aryl, aryl oxy, heteroaryloxy, hetreroaryl, (C₁-C₁₀)alkyl, (C₂-C₁₀)alkenyl, (C₂-C₁₀)alkynyl, (C₃-C₁₀)cycloalkyl, (C₃-C₁₀)heterocycloalkyl, (C₁-C₁₀)alkoxy, amino, CO₂(C₁-C₁₀)alkyl, CO(C₁-C₁₀)alkyl (aryl), (C₁-C₁₀)alkylamino, or
two R substituents attached to the same carbon can be taken together with the carbon to which they are attached to form a cycle selected from (C₃-C₁₀)cycloalkylene, (C₃-C₁₀)heterocycloalkylene, which can be optionally substituted by halo, hydroxy, cyano, (C₁-C₁₀)alkyl, (C₂-C₁₀)alkenyl, (C₂-C₁₀)alkynyl, (C₃-C₁₀)cycloalkyl, aryl;
X is CZ₂-Y, where Z is independently selected from H, halo, cyano, optionally substituted (C₁-C₁₀)alkyl, optionally substituted (C₂-C₁₀)alkenyl, optionally substituted (C₂-C₁₀)alkynyl, optionally substituted (C₃-C₁₀)cycloalkyl,
Y is selected from single bond, CZ₂, O, S, NH, N((C₁-C₁₀)alkyl),
or pharmaceutically acceptable salts thereof, solvates thereof such as, hydrates, with the proviso that the compound of Formula (I) is not meso-1S,2S,1′R,2′R-1-[2-(3,3-dimethyldiaziridin-1-yl) ethyl]-3,3-dimethyldiaziridine (R═Me, X═CH2, n=2).

2. The compound according to claim 1, wherein the compound is selected from the group consisting of:
meso-1S,2S,1′R,2′R-1-[2-(1,2-diazaspiro[12.51]oct-1-yl)ethyl]-1,2-diazaspiro[12.51]octane;
meso-1S,2S,1′R,2′R-1-[3-(3,3-dimethyldiaziridin-1-yl)propyl]-3,3-dimethyldiaziridine;
meso-1S,2S,1′R,2′R-1-[4-(3,3-dimethyldiaziridin-1-yl)butyl]-3,3-dimethyldiaziridine;
meso-1S,2S,1′R,2′R-1-[5-(3,3-dimethyldiaziridin-1-yl)pentyl]-3,3-dimethyldiaziridine;
meso-1S,2S,1′R,2′R-1-[2-(3,3-dimethyldiaziridin-1-yl)ethyl]-1,2-diazaspiro[2.5]octane;
meso-1S,2S,1′R,2′R-1-[2-(1,2-diazaspiro[2.4]hept-1-yl)ethyl]-1,2-diazaspiro[2.4]heptane;
meso-1S,2S,1′R,2′R-1-[2-(3,3-dimethyldiaziridin-1-yl)ethyl]-1,2-diazaspiro[2.4]heptane;
meso-1S,2S,1′R,2′R-1-[2-(1,2-diazaspiro[2.4]hept-1-yl)ethyl]-1,2-diazaspiro[2.5]octane;
meso-1S,2S,1′R,2′R-1-[2-(3-methyl-3-propyldiaziridin-1-yl)ethyl]-3-methyl-3-propyldiaziridine;
meso-1S,2S,1′R,2′R-1-[2-(3-isopropyl-3-methyl-diaziridin-1-yl)ethyl]-3-methyl-3-isopropyldiaziridine;

3. The compound according to claim 1, wherein the compound is selected from the group consisting of:
meso-1S,2S,1′R,2′R-1-[2-(3,3-diethyldiaziridin-1-yl)ethyl]-3,3-diethyldiaziridine;
meso-1S,2S,1′R,2′R-1-[2-(3,3-dipropyldiaziridin-1-yl)ethyl]-3,3-dipropyldiaziridine;
meso-1S,2S,1′R,2′R-1-[2-(3,3-dibutyldiaziridin-1-yl)ethyl]-3,3-dibutyldiaziridine;
meso-1S,2S,1′R,2′R-1-[2-(3,3-dipentyldiaziridin-1-yl)ethyl]-3,3-dipentyldiaziridine;
meso-1S,2S,1′R,2′R-1-[2-(3,3-dihexyldiaziridin-1-yl)ethyl]-3,3-dihexyldiaziridine;
meso-1S,2S,1′R,2′R-1-[2-(3,3-diheptyldiaziridin-1-yl)ethyl]-3,3-diheptyldiaziridine;
meso-1S,2S,1′R,2′R-1-[2-(3,3-dioctyldiaziridin-1-yl)ethyl]-3,3-dioctyldiaziridine;
meso-1S,2S,1′R,2′R-1-[2-(3,3-dinonyldiaziridin-1-yl)ethyl]-3,3-dinonyldiaziridine;
meso-1S,2S,1′R,2′R-1-[2-(3,3-didecyldiaziridin-1-yl)ethyl]-3,3-didecyldiaziridine:
meso-1S,2S,1′R,2′R-1-[2-(3-methyl-3-ethyldiaziridin-1-yl)ethyl]-3-methyl-3-ethyldiaziridine;
meso-1S,2S,1′R,2′R-1-[3-(3,3-diethyldiaziridin-1-yl)propyl]-3,3-diethyldiaziridine;
meso-1S,2S,1′R,2′R-1-[3-(3,3-dipropyldiaziridin-1-yl)propyl]-3,3-dipropyldiaziridine;
meso-1S,2S,1′R,2′R-1-[3-(3,3-dibutyldiaziridin-1-yl)propyl]-3,3-dibutyldiaziridine;
meso-1S,2S,1′R,2′R-1-[3-(3,3-dipentyldiaziridin-1-yl)propyl]-3,3-dipentyldiaziridine;
meso-1S,2S,1′R,2′R-1-[3-(3,3-dihexyldiaziridin-1-yl)propyl]-3,3-dihexyldiaziridine;
meso-1S,2S,1′R,2′R-1-[3-(3,3-diheptyldiaziridin-1-yl)propyl]-3,3-diheptyldiaziridine;
meso-1S,2S,1′R,2′R-1-[3-(3,3-dioctyldiaziridin-1-yl)propyl]-3,3-dioctyldiaziridine;
meso-1S,2S,1′R,2′R-1-[3-(3,3-dinonyldiaziridin-1-yl)propyl]-3,3-dinonyldiaziridine;
meso-1S,2S,1′R,2′R-1-[3-(3,3-didecyldiaziridin-1-yl)propyl]-3,3-didecyldiaziridine;
meso-1S,2S,1′R,2′R-1-[3-(3-methyl-3-ethyldiaziridin-1-yl)propyl]-3-methyl-3-ethyldiaziridine;
meso-1S,2S,1′R,2′R-1-[4-(3,3-diethyldiaziridin-1-yl)butyl]-3,3-diethyldiaziridine;
meso-1S,2S,1′R,2′R-1-[4-(3,3-dipropyldiaziridin-1-yl)butyl]-3,3-dipropyldiaziridine;
meso-1S,2S,1′R,2′R-1-[4-(3,3-dibutyldiaziridin-1-yl)butyl]-3,3-dibutyldiaziridine;
meso-1S,2S,1′R,2′R-1-[4-(3,3-dipentyldiaziridin-1-yl)butyl]-3,3-dipentyldiaziridine;
meso-1S,2S,1′R,2′R-1-[4-(3,3-dihexyldiaziridin-1-yl)butyl]-3,3-dihexyldiaziridine;
meso-1S,2S,1′R,2′R-1-[4-(3,3-diheptyldiaziridin-1-yl)butyl]-3,3-diheptyldiaziridine;
meso-1S,2S,1′R,2′R-1-[4-(3,3-dioctyldiaziridin-1-yl)butyl]-3,3-dioctyldiaziridine;
meso-1S,2S,1′R,2′R-1-[4-(3,3-dinonyldiaziridin-1-yl)butyl]-3,3-dinonyldiaziridine;
meso-1S,2S,1′R,2′R-1-[4-(3,3-didecyldiaziridin-1-yl)butyl]-3,3-didecyldiaziridine;
meso-1S,2S,1′R,2′R-1-[4-(3-methyl-3-ethyldiaziridin-1-yl)butyl]-3-methyl-3-ethyldiaziridine;

4. A pharmaceutically acceptable salt or solvate of the compound of claim 1.

5. A pharmaceutical composition comprising the compound of claim 1 and one or more pharmaceutically acceptable excipients.

6. The pharmaceutical composition of claim 1, wherein the composition comprises a tablet or a capsule.

7. The pharmaceutical composition of claim 4, wherein the composition is administered to a patient in need thereof for the treatment of a mental disorder comprising one or more of adjustment disorders, alcoholism, Alzheimer's disease, anorexia anxious depression, anxiety disorders, bipolar disorder, bulimia, cannabis compound abuse, childhood disorders, cognitive disorders, depression of different origin, depressive disease, dissociative disorders, dysthymia eating disorders, factitious disorders, feeling of reluctance, hypnotic disorder, impulse-control disorders, insomnia, gender identity disorders, labile or saddened mood, major depressive disorder, melancholia, menopause associated depression, mood disorders, obsessive-compulsive disorder, opioid abuse panic disorder, personality disorders, phobic disorders, posttraumatic stress disorder, premenstrual associated depression, primary hypersomnia, primary insomnia, psychosis, psychosis of different origin, psychotic disorders, refractory depression, resistant depression, schizoaffective disorder, schizophrenia, sexual disorders, sleep disorders, somatoform disorders, schizophrenia, substance disorders, suicide intention, suicide, unipolar depression, and unmotivation.

8. The pharmaceutical composition of clam 4, wherein the depression comprises one or more of melancholic depression, severe depression, and psychotic depression.

9. The pharmaceutical composition of claim 4, wherein the psychosis comprises one or more of alcohol psychosis, circular psychosis, involutional psychosis, psychosis associated with dementia, psychosis associated with Alzheimer's disease, psychosis associated with an organic brain syndrome, and drug-induced psychosis.

10. A method of treating a patient in need thereof, the method comprising administering a pharmaceutical composition comprising a therapeutically effective amount of the compound of claim 1 for treating one or more of a mental condition comprising one or more of adjustment disorders, alcoholism, Alzheimer's disease, anorexia anxious depression, anxiety disorders, bipolar disorder, bulimia, cannabis compound abuse, childhood disorders, cognitive disorders, depression of different origin, depressive disease, dissociative disorders, dysthymia eating disorders, factitious disorders, feeling of reluctance, hypnotic disorder, impulse-control disorders, insomnia, gender identity disorders, labile or saddened mood, major depressive disorder, melancholia, menopause associated depression, mood disorders, obsessive-compulsive disorder, opioid abuse panic disorder, personality disorders, phobic disorders, posttraumatic stress disorder, premenstrual associated depression, primary hypersomnia, primary insomnia, psychosis, psychosis of different origin, psychotic disorders, refractory depression, resistant depression, schizoaffective disorder, schizophrenia, sexual disorders, sleep disorders, somatoform disorders, schizophrenia, substance disorders, suicide intention, suicide, unipolar depression, and unmotivation.

11. The method of clam 10, wherein the depression comprises one or more of melancholic depression, severe depression, and psychotic depression.

12. The method of claim 10, wherein the psychosis comprises one or more of alcohol psychosis, circular psychosis, involutional psychosis, psychosis associated with dementia, psychosis associated with Alzheimer's disease, psychosis associated with an organic brain syndrome, and drug-induced psychosis.

13. A compound of formula I′, namely meso-1S,2S,1′R,2′R-1-[2-(3,3-dimethyldiaziridin-1-yl) ethyl]-3,3-dimethyldiaziridine: 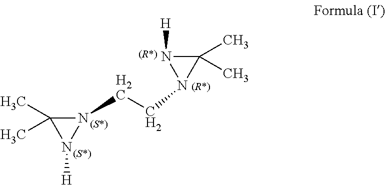
and salts thereof, solvates thereof such as hydrates and any crystal form (including co-crystals and polymorphs) thereof prepared by crystallization of any solvent and pharmaceutical compositions containing such compounds, and use of this compound to treat mental disorders, especially depressions of different etiology, and methods for their preparation.

14. The compound of claim 13 in the form of a pharmaceutically acceptable salt or solvate thereof.

15. The compound of claim 13, wherein the compound is in crystalline form and prepared by crystallization from a solvent.

16. The compound of claim 13 and one or more pharmaceutically acceptable excipients in the form of a pharmaceutical composition.

17. The compound of claim 16, wherein the pharmaceutical composition comprises a tablet or a capsule.

18. The compound of claim 16, wherein the pharmaceutical composition is administered to a patient in need thereof for the treatment of a mental disorder comprising one or more of adjustment disorders, alcoholism, Alzheimer's disease, anorexia anxious depression, anxiety disorders, bipolar disorder, bulimia, cannabis compound abuse, childhood disorders, cognitive disorders, depression of different origin, depressive disease, dissociative disorders, dysthymia eating disorders, factitious disorders, feeling of reluctance, hypnotic disorder, impulse-control disorders, insomnia, gender identity disorders, labile or saddened mood, major depressive disorder, melancholia, menopause associated depression, mood disorders, obsessive-compulsive disorder, opioid abuse panic disorder, personality disorders, phobic disorders, posttraumatic stress disorder, premenstrual associated depression, primary hypersomnia, primary insomnia, psychosis, psychosis of different origin, psychotic disorders, refractory depression, resistant depression, schizoaffective disorder, schizophrenia, sexual disorders, sleep disorders, somatoform disorders, schizophrenia, substance disorders, suicide intention, suicide, unipolar depression, and unmotivation.

19. The compound of claim 16, wherein the depression comprises one or more of melancholic depression, severe depression, and psychotic depression.

20. The compound of claim 16, wherein the psychosis comprises one or more of alcohol psychosis, circular psychosis, involutional psychosis, psychosis associated with dementia, psychosis associated with Alzheimer's disease, psychosis associated with an organic brain syndrome, and drug-induced psychosis.

21. A method of treating a patient in need thereof, the method comprising administering a pharmaceutical composition comprising a therapeutically effective amount of the compound of claim 13 for treating one or more of a mental condition comprising one or more of adjustment disorders, alcoholism, Alzheimer's disease, anorexia anxious depression, anxiety disorders, bipolar disorder, bulimia, cannabis compound abuse, childhood disorders, cognitive disorders, depression of different origin, depressive disease, dissociative disorders, dysthymia eating disorders, factitious disorders, feeling of reluctance, hypnotic disorder, impulse-control disorders, insomnia, gender identity disorders, labile or saddened mood, major depressive disorder, melancholia, menopause associated depression, mood disorders, obsessive-compulsive disorder, opioid abuse panic disorder, personality disorders, phobic disorders, posttraumatic stress disorder, premenstrual associated depression, primary hypersomnia, primary insomnia, psychosis, psychosis of different origin, psychotic disorders, refractory depression, resistant depression, schizoaffective disorder, schizophrenia, sexual disorders, sleep disorders, somatoform disorders, schizophrenia, substance disorders, suicide intention, suicide, unipolar depression, and unmotivation.

22. The method of claim 21, wherein the depression comprises one or more of melancholic depression, severe depression, and psychotic depression.

23. The method of claim 21, wherein the psychosis comprises one or more of alcohol psychosis, circular psychosis, involutional psychosis, psychosis associated with dementia, psychosis associated with Alzheimer's disease, psychosis associated with an organic brain syndrome, and drug-induced psychosis.
